(19) United States
(12) Patent Application Publication
(10) Pub. No.: US 2023/0116922 A1
(43) Pub. Date: Apr. 20, 2023

(54) ORTHOSIS FOR DISTRACTING A HUMAN DIGIT

(71) Applicant: Oxford University Innovation Limited, Oxford (GB)

(72) Inventor: Matthew GARDINER, Oxford (GB)

(21) Appl. No.: 17/910,002

(22) PCT Filed: Mar. 8, 2021
(86) PCT No.: PCT/GB2021/050568

§ 371 Date: Sep. 8, 2022

(30) Foreign Application Priority Data
Mar. 9, 2020 (GB) 2003397.3

(51) Int. Cl. A61F 5/058; A61F 5/048

(52) U.S. Cl. CPC A61F 5/05875; A61F 5/048

(57) ABSTRACT

An orthosis (1) for distracting a human digit that includes a support (6) for removably fitting to a hand or foot and a mounting element (16) for attaching to the distal end of the digit. The orthosis (1) includes an adjustable member (10, 12, 14) extending between the support (6) and the mounting element (16), the adjustable member (10, 12, 14) being physically separate to the mounting element (16). The adjustable member (10, 12, 14) releasably engages with the mounting element (16) and is mounted to the support (6). The adjustable member (10, 12, 14) is movable with the support (6) and the mounting element (16) so to increase the distance between the support (6) and the mounting element (16), which causes the adjustable member (10, 12, 14) to exert a biasing force between the support (6) and the mounting element (16) so to distract the digit.

This invention relates to an orthosis (e.g. splint), in particular to a removable orthosis for distracting a human digit.

Osteoarthritis affects synovial joints in the human body, as a result of the breakdown of joint cartilage and the underlying bone. Treatments to alleviate symptoms include pain killers, removable passive splints, steroid injections and an operation in severe cases.

A more recent advance is the use of joint distraction (separation of the joint), which mechanically “off-loads” the affected joint. This helps return the joint towards its normal biomechanical state and is thought to be disease modifying, rather than just providing symptomatic relief. Enabling natural healing of the joint reduces the number of patients going on to need an operation.

Joint distraction may be performed surgically. However, this is invasive and can result in complications, a prolonged recovery time, and undesired side effects, as well as being expensive. Instead, removable orthoses (e.g. splints) may be used to treat osteoarthritis, such as the first carpometacarpal (CMC) joint, without requiring surgical intervention. However, owing to the discomfort and immobility that patients experience when using such distraction splints, many patients simply discard them and thus do not benefit from their treatment.

For example, conventional thumb splints aim to immobilise the base of thumb, such that the first carpometacarpal (CMC) joint is immobilised, to treat osteoarthritis. However, this is uncomfortable and significantly inhibits use of the thumb, which is normally used for many different tasks every day.

It is an aim of the present invention to provide an improved distraction orthosis for a human digit.

When viewed from a first aspect the invention provides an orthosis for distracting a human digit, the orthosis comprising:
a support for removably fitting to a hand or foot comprising the digit;
a mounting element for attaching to the distal end of the digit; and
an adjustable member extending between the support and the mounting element;
wherein the adjustable member and the mounting element are physically separate components;
wherein the adjustable member is arranged to releasably engage with the mounting element;
wherein the adjustable member is mounted to the support;
wherein the adjustable member is movable with respect to one or both of the support and the mounting element so to increase the distance between the support and the mounting element; and
wherein the orthosis is arranged such that the increase in distance between the support and the mounting element causes the adjustable member to exert a biasing force between the support and the mounting element so to distract the digit.

The present invention relates to a human digit distraction orthosis (e.g., and henceforth referred to as, a splint). The splint includes three main components: a support that can be removably fitted to (e.g. a part of) the hand or foot comprising the digit to be distracted, a mounting element that is to be attached to the distal end of the digit (e.g. the opposite end of the digit to which the support is fitted), and an adjustable member that extends between the support and the mounting element.

The support is configured to fit to the hand or foot including a digit to be distracted, so it can be worn by a user. The hand or foot also provides a surface on which the support can rest to oppose the biasing force applied against the support by the adjustable member. The support is arranged such that it may be removed from the digit, e.g. after treatment.

The mounting element is configured to be attached to the distal (opposite) end of the digit from the base of the digit, again in a way that allows it to oppose the biasing force applied by the adjustable member.

It will be appreciated that the anatomy of a human hand (or foot) provides these two anchor points for the splint and thus a splint of this type is particularly suited for distraction of a digit and the treatment of osteoarthritis. The splint is also suited for treatment of a bone fracture or ligamentous injury, e.g. using similar distraction of the digit.

The adjustable member is configured to be moved (i.e. adjusted) relative to the support and/or the mounting element (e.g. the adjustable member comprises an adjustment mechanism). This allows the splint, in use (when affixed to a user's digit), to increase the distance between the support and the mounting element. The increase in distance applies a biasing force between the support and the mounting element, i.e. biasing them away from each other.

The biasing force thus acts to distract the digit on which the splint is being used. In this way, the distraction splint of the present invention can be used to relieve the symptoms of osteoarthritis and enable natural healing of the joint to occur, by mechanically “off-loading” an osteoarthritic joint in the digit being distracted. When being used for fracture management, the distraction force helps to treat the fractured bone, e.g. by using the principal of ligamentotaxis to draw the bone ends together.

The adjustable member and the mounting element are physically separate components and the adjustable member is configured to be engage in a releasable manner with the mounting element, i.e. the adjustable member can be separated from the mounting element. Being able to separate the adjustable member and the mounting element (and thus preferably the mounting element and the support from each other) helps to allow the splint to be disassembled and thus at least partially be removed from a user's hand or foot. The splint may subsequently be reassembled and refitted to the user's hand or foot.

It will be appreciated that by providing a distraction splint which can be easily disassembled helps to improve a user's comfort when wearing the splint. For example, when the splint is disassembled (and, e.g., removed), this may provide the user with increased dexterity to perform day-to-day tasks and, e.g., allow the user's hand or foot to be washed (along with the splint itself). This increases the likelihood that a user will continue to wear the splint, e.g. to treat their thumb base osteoarthritis. The treatment provided by the splint may thus help to reduce the rate in patients at which replacement of the thumb joint becomes necessary.

The splint may be configured to distract any suitable and desired human digit, on the hand or foot. Preferably the splint is arranged to distract a digit on a hand, i.e. a finger or thumb. It will be appreciated that the anatomy of the hand is suited to providing an anchor point for the support of the splint, e.g. at the base of the fingers. The anatomy of the thumb is particularly suited to be distracted by the splint of the present invention, owing to the generally smaller size of the thumb and the presence of the palm of the hand at the base of the thumb, which may provide a convenient anchor point for the support of the splint.

The splint may be arranged to distract the digit for any particular use, e.g. to treat any suitable and desired condition. In one embodiment the splint is used to distract a human digit for treating osteoarthritis. In one embodiment is used to distract a human digit for treating a fractured digit.

The splint may be configured to distract any suitable part (e.g. joint) of the digit that on which it is being worn. In a preferred embodiment the splint is used to distract a carpometacarpal (CMC) joint (when being used on the thumb), a metacarpophalangeal or interphalangeal joint (when being worn on a finger) or a metatarsophalangeal (MTP) or interphalangeal joint (when being used on a foot digit).

The support of the splint, for removably fitting to the hand or foot including the digit on which the splint is to be worn, provides a platform on which the rest of the splint is constructed, i.e. to which the adjustable member is attached. The support also provides a platform through which the biasing force is applied to the proximal end of the digit, e.g. to distract the CMC, MCP or MTP joint.

The support may be configured to fit removably to the hand or foot on which the splint is to be worn in any suitable and desired way. In a preferred embodiment the support is arranged to be fitted non-surgically to the hand or foot.

The support may be arranged to removably fit to any suitable and desired part of the hand or foot comprising the digit to be distracted. In one embodiment the support may be arranged to be removably fitted to the wrist of a hand or the ankle of a foot. In a preferred embodiment the support is configured to fit removably to the base of the digit on which the splint is to be worn. The base of a digit provides a particularly convenient location (and surface) on which the support may rest.

Preferably the support is arranged (e.g. shaped) to rest on the outside of (i.e. the skin at) the base of the digit. This helps to allow the support to be removed easily from the user's digit, e.g. when the adjustable member is disengaged from the mounting element. Thus the bias force acting through the support preferably acts on the outside of (i.e. the skin at) the base of the digit.

The (e.g. support of the) splint may be arranged (e.g. shaped) such that the splint is removably fitted to any suitable and desired part of the hand or foot, e.g. at the base of the digit. In a preferred embodiment the support is arranged (e.g. shaped) for removably fitting proximal to (e.g. around) the metacarpophalangeal (MCP) joint (when the splint is being used on a hand digit) or the metatarsophalangeal (MTP) joint (when the splint is being used on a foot digit).

In a preferred embodiment the support comprises a sheath, e.g. for removably fitting around the hand or foot, e.g. around the base of the digit. Thus preferably the support is shaped to extend (e.g. substantially fully) around the hand or foot, e.g. around the base of the digit. For example, preferably the support comprises an aperture for receiving the hand or foot, or the digit, therethrough. This allows the support to be removably fitted onto the hand or foot, or the digit, and helps the support to remain securely in place while the splint is being used.

In one embodiment the support is shaped to extend (e.g. substantially fully) around the (e.g. base of the) hand or foot. This may allow the support to be fitted more securely.

Preferably the support has a length that is at least partially extended for fitting along the palm and/or back of the hand (or the sole and/or top of the foot), e.g. extending below the MCP or MTP joint. A support that has an extended length (e.g. compared to a thinner ring around the base of the digit) may help to increase the comfort of the splint for the user, e.g. owing to the biasing force being applied through the support being distributed over a larger surface area that the support contacts the digit.

Preferably the support is extended from the location (or locations) at which the adjustable member is mounted on the support, in a direction away from the direction in which the adjustable member extends towards the mounting element. Preferably the support does not extend along the digit itself, e.g. above the MCP or MTP joint. This helps to allow the support to provide a suitable surface over which the biasing force acts, while helping to keep the digit free and not, for example, covering the MCP or MTP joint so to avoid applying pressure to this joint. Again, this helps to improve the comfort of the splint for the user, as it may allow the digit to be used while the splint is being worn.

In one embodiment the support is shaped such that, when the splint is fitted to the digit, the support leaves the palm of the hand (or the sole of the foot) substantially uncovered. Again, this this helps to improve the comfort of the splint for the user, as it may allow the hand (or foot) to be held in a relaxed position while the splint is being worn.

The support may be shaped to provide some space between the support and some of the part of the hand or foot on which it rests, so not to restrict blood vessels.

In one embodiment the (e.g. inner surface of the) support is shaped to conform to the shape of the digit (and, e.g., the part of the associated hand or foot) to which the splint is to be fitted. Again, this helps to improve the comfort of the splint for the user. The support may be shaped to conform to the shape of the digit in any suitable and desired way. For example, different splints could be made (e.g. with the support made) in a variety of different (e.g. shapes and) sizes, and the most suitable size (and/or shape for a user's digit may be chosen. Left and right handed (or footed) versions may be provided, as appropriate.

In one embodiment the support is at least partially made from a material that is flexible, e.g. mouldable. This helps the support to conform to the shape of a user's digit (and, e.g., the part of the associated hand or foot) when it is worn. However, it will be appreciated that preferably the part of the support, on which the adjustable member is mounted, is substantially rigid. This helps to apply the biasing force effectively to the support to distract the digit.

In one embodiment the support is shaped to be customised to the particular shape of a user's digit (and, e.g., the part of the associated hand or foot) when it is worn. This may be achieved by at least partially making the support from a material that is mouldable (but which, for example, may set into a rigid form once moulded). In one embodiment the support is individually moulded or 3D printed. Thus, for example, the digit (and, e.g., the part of the associated hand or foot) on which the splint is to be worn may be (e.g. 3D) scanned. The support may then be produced using the scan, as appropriate.

In one embodiment the support is at least partially made from a material that is elastic. This may help the support to be stretched over and to grip onto the digit on which it is being worn, e.g. when the support extends around the hand or foot, as well as to conform to the digit and/or hand or foot.

In one embodiment the support comprises a plurality of (e.g. ventilation) holes across the surface of (and extending through) the support, e.g. the support may comprise a mesh. This may help to allow air to reach the user's skin to help prevent the skin perspire, thus again increasing the comfort of the splint when being worn.

The support may be made from any suitable and desired material, e.g. depending on how it is manufactured. In one embodiment the support comprises (is made from) a plastic material.

The mounting element, for attaching to the distal end of the digit on which the splint is to be worn, is configured to be attached to the distal (opposite) end of the digit from the support. The mounting element thus provides another platform (in addition to the support) on which the splint is based, in a way that allows the biasing force to be applied by the adjustable member in order to distract the digit.

The mounting element may be configured to be attached to the distal end of the digit on which the splint is to be worn in any suitable and desired way. In a preferred embodiment the mounting element is arranged to be attached non-surgically to the distal end of the digit (e.g. no alteration or invasive procedure is required to the digit to attach the mounting element). Thus preferably the whole of the splint (i.e. both the support and the mounting element) is arranged to be attached or fitted to the digit of the user non-surgically.

The mounting element may be configured to be attached to any suitable and desired part of the distal end of the digit. Preferably the mounting element is configured to be attached to the nail of the digit. It will be appreciated that the nail provides a convenient fixing point for attaching the mounting element, owing to its location and its relatively smooth surface and rigid structure (compared, for example, to the surrounding tissue and skin).

Thus preferably the mounting element comprises a (e.g. concave) surface for attaching to the nail of the digit. Preferably the mounting element (e.g. the surface of the mounting element for attaching to the nail of the digit) has a size (e.g. a surface area) that is less than or equal to the size of the nail of the digit to which it is to be attached. For example, preferably the mounting element has a maximum dimension less than 2 cm.

In one embodiment the mounting element comprises a plate (e.g. comprising a concave surface) for attaching to the nail of the digit. Preferably the mounting element is arranged to not encircle the digit to which it is attached, but instead, for example, is simply attached to the nail of the digit. Preferably the mounting element is attached to the nail using an adhesive, e.g. a glue. Preferably the mounting element (and preferably all the components of the splint) are arranged to be attached to (or mounted on) the digit non-surgically.

In some embodiments the mounting element comprises (e.g. is made from) a pliable material. In some embodiments the mounting element may (comprise a material such that it is able to) be shaped (e.g. trimmed) to any suitable and/or desirable shape. This may help it to be arranged to attach a desired portion (e.g. nail) of the digit. For example, the mounting element may be trimmed such that the size and shape of the mounting element is complementary to the nail of the digit, e.g. the mounting element substantially fits within the nail of the digit, e.g. with minimal overhang of the material outside the nail of the digit.

It will be appreciated that the mounting element, in at least preferred embodiments, may be a relatively small component (e.g. compared to the rest of the splint and to the digit) that simply allows the rest of the splint (via the adjustable member) to be attached to the mounting element, to thus apply the biasing force between the distal end of the digit and the part of the hand or foot on which the support is fitted, e.g. at the base of the digit. By allowing the adjustable member to be removably attached to the mounting element, the distal end of the digit can be freed (e.g. temporarily) from the rest of the splint, thus allowing the digit to be used to perform more dextrous tasks. The detachment of the adjustable member from the mounting element may also allow the rest of the splint to be removed from the digit, thus allowing the digit (and, e.g., the rest of the hand or foot) to be washed.

The adjustable member extends between the support and the mounting element, is mounted to the support and releasably engages with the mounting element. The adjustable member is mounted to the support and engages with the mounting element in a way that allows the adjustable member to apply a biasing force between the support and the mounting element, biasing them away from each other and thus acting in use to distract the digit.

The adjustable member may extend between the support and the mounting element, in order to connect the support and the mounting element, in any suitable and desired way. In one embodiment the adjustable member comprises a (e.g. load bearing) frame that extends between the support and the mounting element. The adjustable member may, for example, comprise one or more rods that extend between the support and the mounting element or a sheath for at least partially extending around the digit between the support and the mounting element.

In a preferred embodiment the adjustable member comprises two rods that extend (e.g. either side of a void into which the digit on which the splint is to be placed is inserted) between the support and the mounting element.

In one embodiment the load bearing structure of the adjustable member (i.e. its ability to apply a biasing force between the support and the mounting element) may be provided separately from the movable part (e.g. an adjustment mechanism) of the adjustable member (i.e. its ability to increase the distance between the support and the mounting element). However, in another embodiment the part of adjustable member through which the biasing force is applied is movable (e.g. comprises the adjustment mechanism) with respect to one or both of the support and the mounting element (so to increase the distance between the support and the mounting element).

Thus, in one set of embodiments, the frame, rod(s) or sheath are both load bearing and movable with respect to the support and the mounting element. For example, the rod(s) or sheath may comprise an (e.g. integral) adjustment mechanism, such as a screw thread, ratchet or soft robotics.

The adjustable member could be made from any suitable and desired material. The adjustable member may be made from (e.g. moulded) plastic (e.g. when the adjustable member comprises a sheath) or from metal (e.g. when the adjustable member comprises one or more rod(s)).

The adjustable member and the mounting element are physically separate components and the adjustable member is configured to be engage in a releasable manner with the mounting element, so that the adjustable member can be separated from the mounting element. Thus the adjustable member and the mounting element are non-integral components that engage (mechanically interact) with each other.

The adjustable member and the mounting element may engage releasably with each other in any suitable and desired way, e.g. such that when engaged the adjustable member is able to act on the mounting element to apply a bias thereto.

In one embodiment the adjustable member and the mounting element comprise respective complementary engaging surfaces, wherein the adjustable member and the mounting element are arranged to releasably engage with each other at the complementary engaging surfaces. Preferably the bias is applied through the complementary engaging surfaces.

The complementary engaging surfaces may simply comprise respective flanges (e.g. abutting surfaces) on the adjustable member and the mounting element that abut each other when the adjustable member and the mounting element engage. In one embodiment the adjustable member and the mounting element are arranged to be releasably fastened to each other. Thus, for example, when the adjustable member and the mounting element are fastened to each other, the adjustable member and the mounting element may require a retaining force to be overcome to separate them (as opposed, for example, to abutting surfaces that may simply be moved apart without any retaining force needing to be overcome). This helps to retain the adjustable member and the mounting element engaged with each other while in use.

The adjustable member and the mounting element may be releasably fastened to each other in any suitable and desired way. In one embodiment the adjustable member and the mounting element comprise complementary releasable fasteners, such as a clip, a slot, a detent or a latch. For example, the mounting element may comprise a clip or a slot and the adjustable member may comprise a (e.g. beaded) flange or a projection for releasably fastening in the clip or into the slot respectively. In one embodiment the (e.g. engaging surface and/or the releasable fastener of the) mounting element is compliant or flexible (e.g. is made from a compliant or flexible material). This may aid the engagement of the adjustable member with the mounting element and help to retain them in this position. In a preferred embodiment the mounting element is made from (e.g. a compliant or flexible) plastic.

In one embodiment the mounting element comprises an engaging portion that engages with the adjustment mechanism of the adjustable member. For example, the mounting element may comprise part of a ratchet that engages with the other part of the ratchet on the adjustable member.

The adjustable member and the mounting element (and, for example, the complementary engaging surfaces and/or releasable fasteners) may be arranged relative to each other such that the biasing force applied by the adjustable member to the mounting element is a tensile force (i.e. pulling on the mounting element) or a compressive force (i.e. pushing on the mounting element). Preferably the (e.g. complementary engaging surfaces and/or releasable fasteners of the) adjustable member and the mounting element are arranged such that the biasing force acts in an opposite direction to the direction in which the adjustable member and the mounting element are (moved to be) released from each other.

In one embodiment the biasing force applied, in use, between the support and the mounting element, acts to retain the adjustable member and the mounting element engaged with each. This may help to simplify the design of the engaging parts of the adjustable member and the mounting element, as specific features to retain them together may not be necessary.

Preferably the (e.g. complementary engaging surfaces and/or releasable fasteners of the) adjustable member and the mounting element are arranged such that the support is able to be removed from the digit when the adjustable member and the mounting element are released (separated) from each other.

As indicated above, the load bearing structure of the adjustable member may be provided separately from the movable part of the adjustable member. Thus, in one embodiment, the complementary engaging surfaces and/or releasable fasteners are independent of the movable part of the adjustable member, e.g. the movable part of the adjustable member is provided in a different part of the adjustable member to the complementary engaging surfaces and/or releasable fasteners.

For example, when the adjustable member comprises two rods that extend between the support and the mounting element, preferably the adjustable member comprises a bar that extends (and is connected) between the two rods, wherein the bar is arranged to releasably engage with (e.g. clip into) the (e.g. clip of the) mounting element. Preferably the bar comprises two apertures through which the two rods pass respectively. This allows the bar to be connected to the two rods.

The bar may releasably engage with the mounting element in any suitable and desired manner. In one embodiment the bar comprises a longitudinal portion that extends between the two rods, wherein the longitudinal portion is arranged to releasably engage with (e.g. clip into) the (e.g. clip of the) mounting element. In one embodiment the bar comprises a projection (e.g. from the longitudinal portion) in the direction of the mounting element, wherein the projection is arranged to releasably engage with (e.g. slot into) the (e.g. slot of the) mounting element. When the mounting element is compliant or flexible, this may help the projection of the bar to slot into the mounting element.

In some embodiments, the mounting element comprises a recess arranged to receive a portion of the adjustable member (e.g. a portion of the bar). For example, the mounting element may comprise a recess arranged to receive a projection (e.g. from the longitudinal portion) of the bar in the direction of the mounting element, e.g. the projection of the bar slots into the projection of the mounting element. In some embodiments the bar may be fixedly engaged (e.g. secured in place) in the recess of the mounting element. In some embodiments the bar may be releasably engaged (e.g. slot into) the (e.g. the recess) of the mounting element.

Preferably the adjustable member (e.g. the rods and the bar) are arranged such that the position of the bar is adjustable. For example, the bar may be movable relative to the rods, such that it can adjust the distance between the mounting element and the support. Preferably the rods and the bar are arranged such that the adjustment of the bar by each rod is independent of the other rod.

The adjustable member may be mounted to the support in any suitable and desired way, such that the biasing force can be applied between the adjustable member and the support. Preferably the adjustable member is anchored to the support.

Preferably the adjustable member is mounted (e.g. anchored) to the support such that longitudinal movement of (e.g. at least part of) the adjustable member (in the direction between the support and the mounting element, e.g. in use of the splint) is substantially prevented. For example, when the adjustable member comprises one or more rods, preferably longitudinal movement of the one or more rods relative to the support is substantially prevented. This helps the adjustable member to apply the biasing force between the support and the mounting element.

Preferably the adjustable member is pivotably mounted (e.g. anchored) to the support. This allows some lateral movement of the adjustable member distal from the support, which may allow the wearer of the splint some freedom to move (e.g. flex) their digit while wearing the splint and/or the adjustable member to be released from the mounting element.

In some embodiments, the adjustable member comprises two rods, wherein each rod comprises a screw thread and an integral knob (e.g. ball) located at one end of the rod. Preferably the knob of the (e.g. each) rod is proximate to the support, such that the rod of the adjustable member is pivotally mounted (e.g. anchored) to the support via the knob. For example, the support may be (e.g. comprise two recesses) arranged to receive the knobs of the rods respectively, such that the knobs and (e.g. the recesses of the) support are pivotally engaged (e.g. a ball-and-socket joint is formed between (each of) the rods and the support), while longitudinal movement of (e.g. a least part of) the rod of the adjustable member is substantially prevented.

The adjustable member is configured to be moved (i.e. adjusted in position) relative to the support and/or the mounting element, such that the distance between the support and the mounting element can be increased. This allows the adjustable member to apply the biasing force between the support and the mounting element. Preferably the splint is arranged such that the adjustable member is movable so to increase the length of the adjustable member between the support and the mounting element (to increase the distance between the support and the mounting element).

The adjustable member may be movable (adjusted) in any suitable and desired way to increase the distance between the support and the mounting element. In one embodiment the adjustable member is slidable with respect to one or both of the support and the mounting element so to increase the distance between the support and the mounting element.

In a preferred embodiment (e.g. at least a part of) the adjustable member is rotatable with respect to one or both of the support and the mounting element so to increase the distance between the support and the mounting element. Preferably (e.g. at least a part of) the adjustable member is rotatable about its longitudinal axis (in the direction between the support and the mounting element, e.g. in use of the splint).

In one set of embodiments the (e.g. adjustment mechanism of the) adjustable member comprises a screw thread or ratchet. In one embodiment the adjustable member comprises two or more (e.g. concentric) parts having complementary screw threads or ratchets, wherein the two or more parts are rotatable or movable relative to each other to increase the distance between the support and the mounting element. In one embodiment, one or both of the support and the mounting element comprises a complementary screw thread or ratchet arranged to increase the distance between the support and the mounting element when the (e.g. screw thread or ratchet of the) adjustable member is rotated or moved relative to one or both of the support and the mounting element.

In one embodiment the adjustable member is indexed (e.g. the adjustable member comprises a ratchet). This helps the adjustable member to be retained in a position to which it has been moved (e.g. when increasing the distance between the support and the mounting element). It also helps to give an indication of the distance through which the adjustable member has been moved (and thus the corresponding increase in distance between the support and the mounting element), as the indexing of the adjustable member may (and preferably does) correspond to a particular increase in the distance between the support and the mounting element).

The adjustable member may be actuated to be moved, so to increase the distance between the support and the mounting element when the adjustable member is engaged with the mounting element, in any suitable and desired way. In one embodiment the splint comprises an actuator for actuating the adjustable member. Any suitable and desired actuator may be provided, e.g. depending on the manner in which the adjustable member is to be moved (e.g. slid or rotated). The actuator may be an integral part of the adjustable member, the support or the mounting element, or may be removable. This latter option may allow the actuator to be fitted for ease of moving the adjustable member and then to be removed during other use of the splint.

In one embodiment the actuator (e.g. of the adjustable member, the support or the mounting element) comprises a complementary screw thread or ratchet to the screw thread or ratchet of the adjustable member. This allows the actuator to be moved relative to the screw thread or ratchet of the adjustable member. Thus, for example, one or more of the adjustable member, the support and the mounting element may comprise a nut (e.g. comprising a screw thread) arranged to be actuated to increase the length of the adjustable member between the support and the mounting element.

In one embodiment the actuator (e.g. of the adjustable member, the support or the mounting element) is fixedly attached to (e.g. screw thread or ratchet of) or an integral part of the adjustable member. This allows the actuator to be moved (e.g. turned) to actuate the screw thread or ratchet of the adjustable member, e.g. relative to a complementary screw thread or ratchet on a different part of the (e.g. adjustable member of the) splint.

In one embodiment the screw thread of the adjustable member comprises the actuator, e.g. formed as an integral part of the screw thread. Preferably the actuator is located at one end of the screw thread, e.g. distal from the support and proximal to the mounting element.

The actuator may, for example, comprise a male-type fitting such as a knob (e.g. a handle, grip, lever or (e.g. winged) nut) or a female-type fitting such as a socket (e.g. to be actuated using a screwdriver or (e.g. Allen) key). The actuator (e.g. knob or socket) may be actuated by hand or by a tool (e.g. spanner, screwdriver or (e.g. Allen) key).

When the adjustable member comprises two rods that extend between the support and the mounting element, preferably each of the rods is (e.g. independently) movable and, e.g., actuatable. Providing parts of the adjustable member that are independently movable helps to allow a differential distraction to be provided by the splint and thus greater control over the treatment of the digit on which the splint is being used.

Thus, for example, when the adjustable member comprises two rods having screw threads (or ratchets), preferably the adjustable member comprises two knobs (e.g. nuts) (mounted (e.g. fixedly) on the two rods respectively) or two (e.g. hex) sockets (formed (e.g. integrally) in the two rods respectively) for actuating (rotating or sliding) the (two rods respectively of the) adjustable member to increase the distance between the support and the mounting element. When the adjustable member comprises a bar (for engaging with the mounting element) that extends between the two rods, preferably the two knobs or sockets are located on the side of the bar that is remote from the support (e.g. at the ends of the rods that are distal from the support and proximal to the mounting element).

Preferably the bar comprises complementary screw threads or ratchets (e.g. defined in the two apertures in the bar through which the two rods pass respectively) for adjusting the position of the bar relative to the rods. The complementary screw threads or ratchets may, for example, be provided by a nut or fitting that is formed (e.g. attached) in the (e.g. two apertures of the) bar. Preferably the rods are independently actuatable (e.g. rotatable or slidable), relative to the bar, to adjust the position of the bar differentially relative to the rods.

In some embodiments, the complementary screw threads may be integrally formed in the bar. For example, in some embodiments the bar may comprise two apertures extending through the bar material (e.g. one on each side of the bar), wherein each aperture comprises an integrally formed (e.g. complementary) screw thread that is complementary to the screw thread of the rods of the adjustable member. Thus, rotation of the rods through the apertures may allow adjustment of the position of the bar with respect to the rods and thus the support.

When the adjustable member is adjusted, to increase the distance between the support and the mounting element, this exerts a biasing force between the support and the mounting element. The adjustable member may be arranged in any suitable and desired way to exert the biasing force. Preferably the adjustable member is load bearing, e.g. substantially rigid.

Preferably the splint is arranged such that the biasing force is applied in the direction between the support and the mounting element, e.g. through the (length of the) adjustable member.

Preferably the splint is arranged such that the biasing force is applied to distract a CMC joint (e.g. of the thumb) or a MTP joint.

In one embodiment the splint comprises a sensor arranged to determine a parameter representative of the biasing force and/or of the distance between the support and the mounting element. This helps to more accurately control the distraction of the digit.

In one embodiment the sensor comprises a Hall effect sensor. Preferably the Hall effect sensor is arranged to measure a parameter representative of the biasing force between the support and the mounting element. The Hall effect sensor may, for example, be mounted on the support or the adjustable member. The Hall effect sensor may detect deformation of the support or the adjustable member, e.g. to measure the parameter representative of the biasing force between the support and the mounting element.

In some embodiments (a component of) the splint (e.g. the bar) may comprise a transparent portion (e.g. window) which may be used to measure (e.g. by eye or via a sensor) the biasing force and/or of the distance between the support and the mounting element.

In some embodiments the components of the distraction splint may be formed by any suitable and/or desirable method. For example, the support, adjustable member, rods and/or bar may be individually moulded or 3D printed, or any desirable combination thereof. It will be appreciated that there is no requirement for all of the components of the distraction splint to be made using the same technique.

Certain preferred embodiments for the invention will now be described, by way of example only, with reference to the accompanying drawings in which:

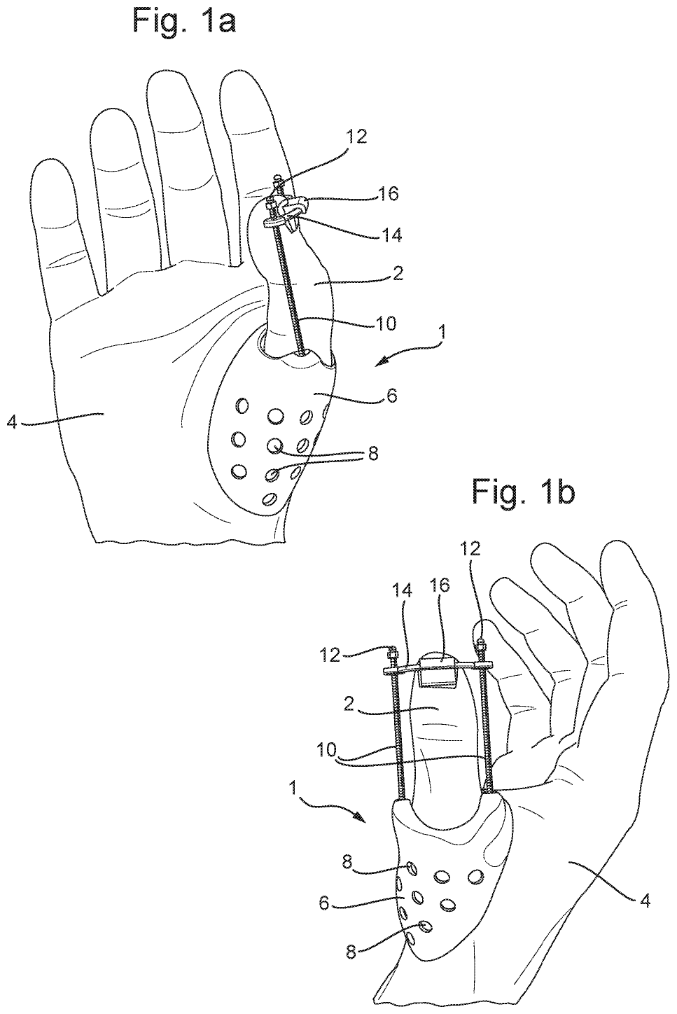

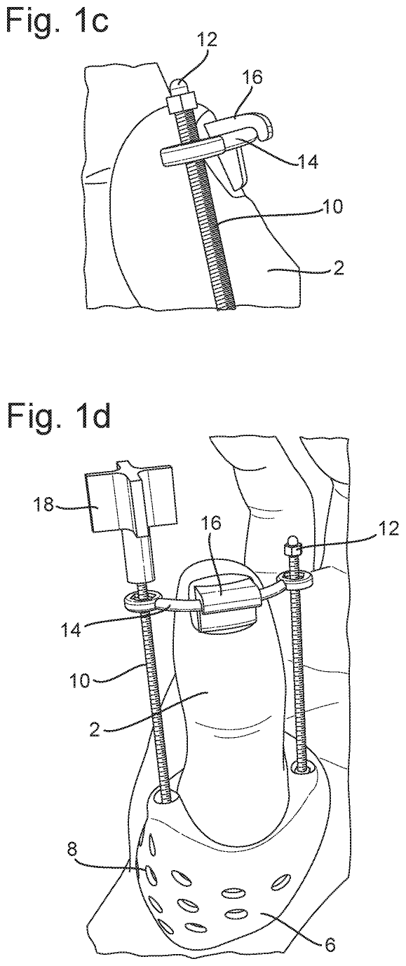

FIGS. 1a, 1b, 1c and 1d show views of a distraction splint according to an embodiment of the present invention;

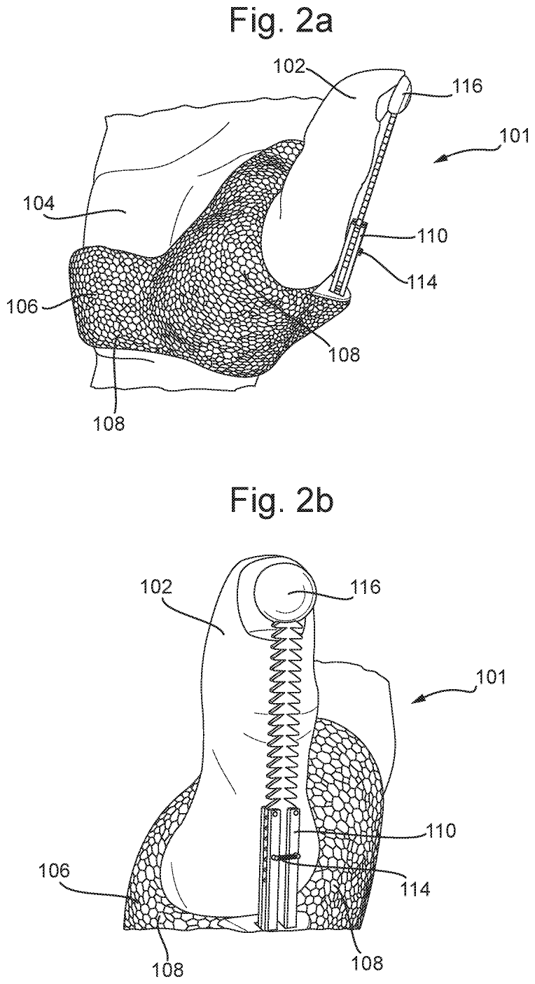

FIGS. 2a and 2b show views of a distraction splint according to another embodiment of the present invention;

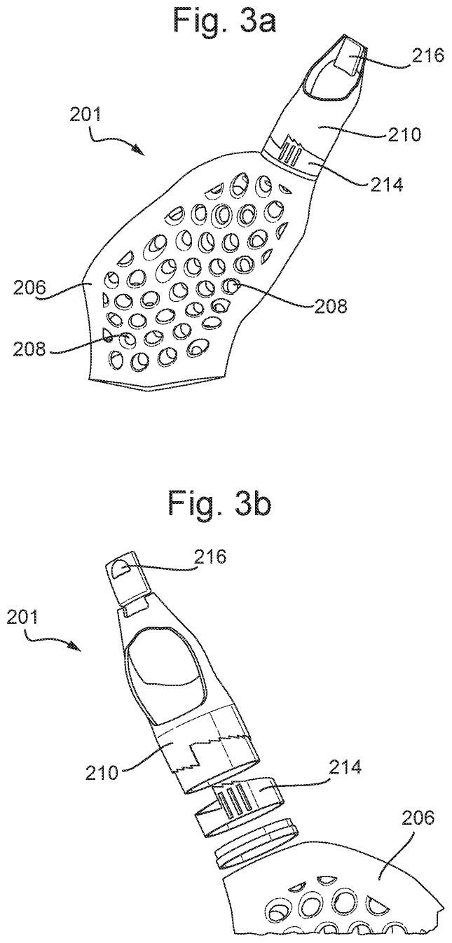

FIGS. 3a and 3b show views of a distraction splint according to another embodiment of the present invention;

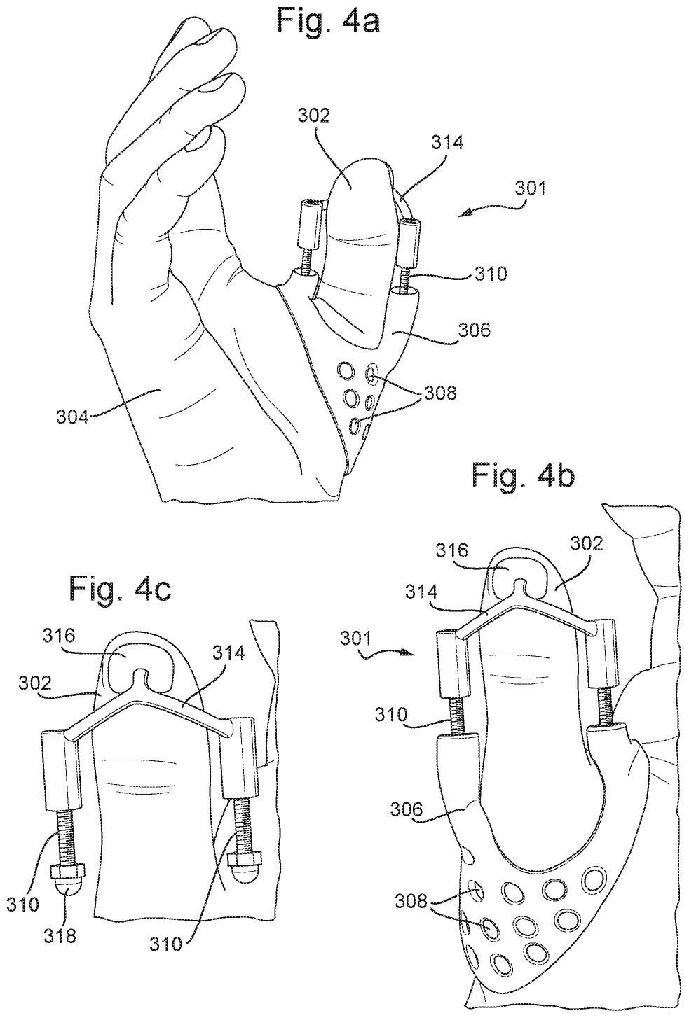

FIGS. 4a, 4b and 4c show views of a distraction splint according to another embodiment of the present invention;

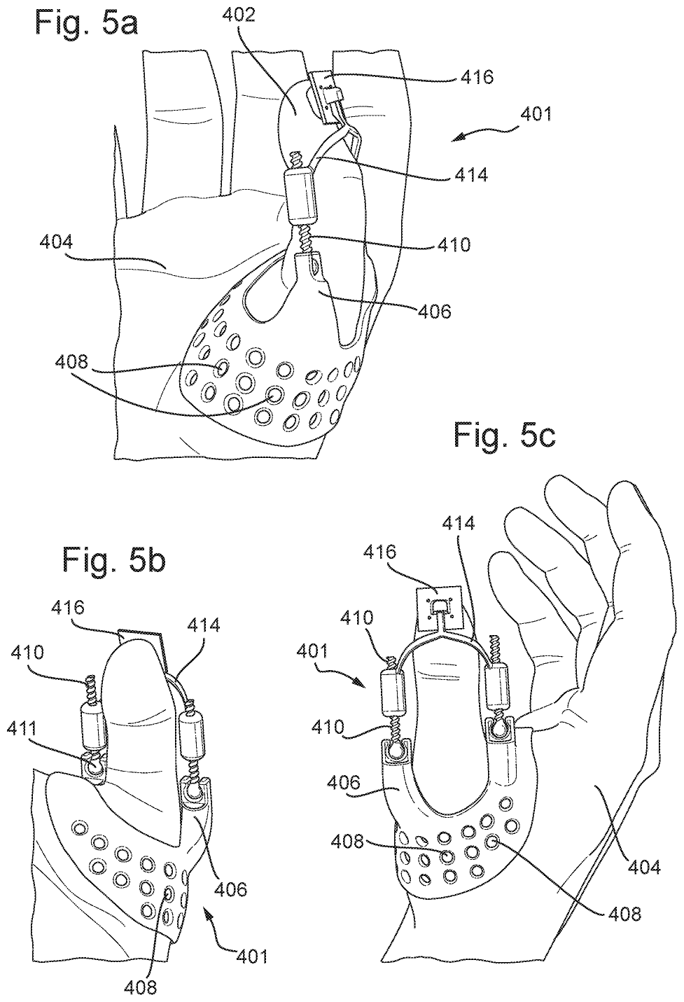

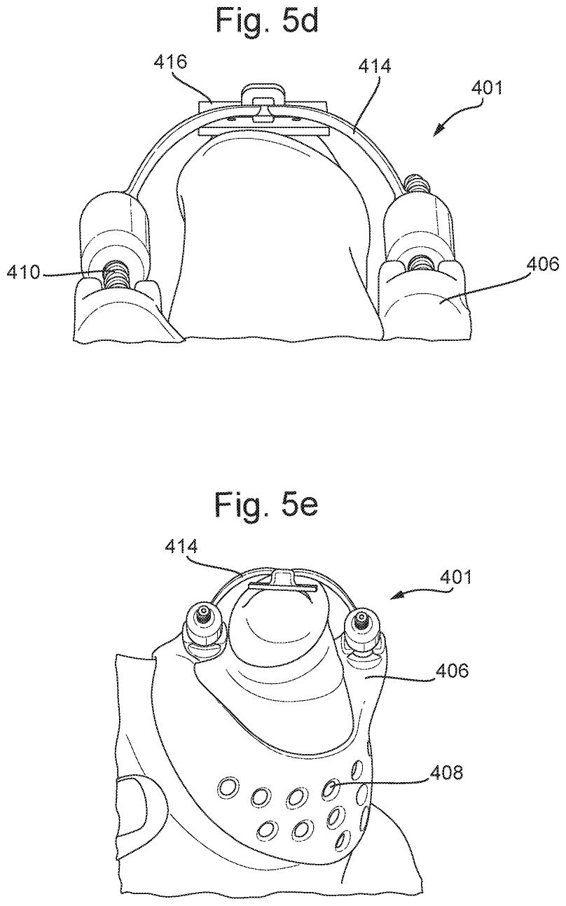

FIGS. 5a, 5b, 5c, 5d and 5e show views of a distraction splint according to another embodiment of the present invention;

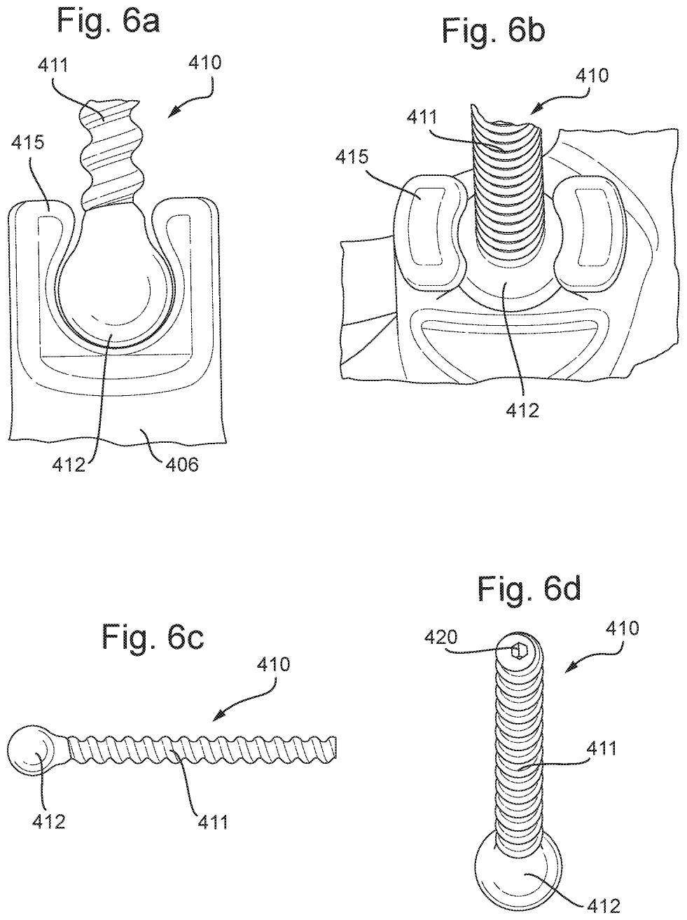

FIGS. 6a, 6b, 6c and 6d show views of a distraction splint according to another embodiment of the present invention;

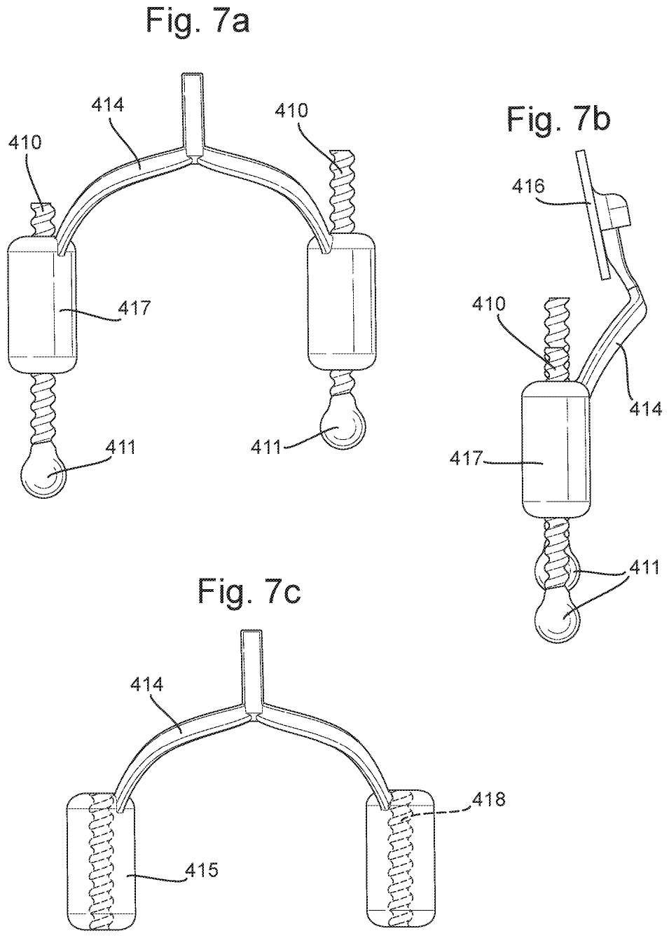

FIGS. 7a, 7b and 7c show views of the screws and wishbone for use in accordance with some embodiments of the present invention; and

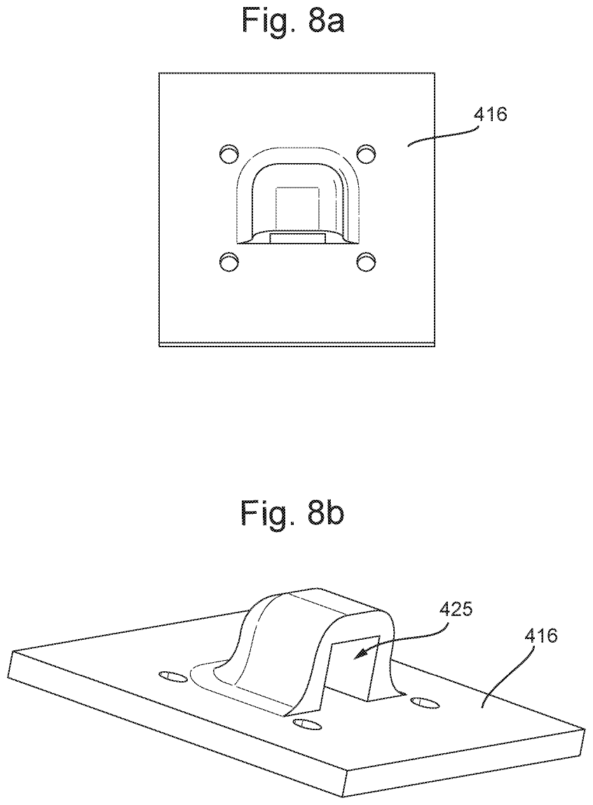

FIGS. 8a and 8b show views of a mounting element for use in accordance with some embodiments of the present invention.

Distraction splints may be used to treat joint conditions, such as osteoarthritis, by distracting the affected joint to allow healing to occur. Distraction splints in accordance with embodiments of the invention, which may be used to treat conditions such as osteoarthritis or a fracture, will now be described.

FIGS. 1a, 1b, 1c and 1d show views of a distraction splint 1 according to an embodiment of the present invention. The distraction splint 1 shown in FIGS. 1a, 1 b, 1c and 1d is designed to be fitted to and worn on a thumb 2 of a hand 4 of a person being treated with the splint 1.

It will be seen from FIGS. 1a, 1 b, 1c and 1d that the distraction splint 1 is designed for, and is being worn on, the right hand 4 of the person being treated. It will be appreciated that a distraction splint 1 for a left hand may also be provided, based on this same design (e.g. a mirror image thereof), according to an embodiment of the present invention.

The distraction splint 1 comprises a support 6 that removably fits around the base of the thumb 2, around the metacarpophalangeal (MCP) joint. The support 6 is moulded (e.g. to fit the hand 4 of the person) from plastic (e.g. suitable for 3D printing with) and is shaped to rest on the palm of the hand 4 at the base of the thumb 2.

The support 6 has multiple holes 8 formed through the thickness of the support 6. The holes 8 provide some ventilation to help prevent the support 6 causing the hand 4 to become sweaty.

A pair of adjustable rods 10 is mounted on the support 6 either side of the thumb 2. The rods 10 each comprise a screw thread. The rods 10 are each mounted pivotably to the support 6 in a way that prevents them from being moved substantially longitudinally (in a direction away from the base of the thumb 2). This helps to prevent the rods 10 from being extracted from the support 6 and from moving further into the support 6. The rods 10 are also mounted such that they are able to rotate freely (in the rotational direction the screw threads).

The other ends of the rods 10 (remote from the support 6) comprise a pair of nuts 12 that is fixedly mounted to the pair of rods 10 respectively. The nuts 12 are attached to the rods 10 in a way that allows the nuts 12 to be rotated to rotate the respective rods 10. As shown in FIG. 1d, a removable tool 18 may be attached to the nut 12 to aid adjustment.

A bar 14 is mounted on and between the pair of rods 10, between the support 6 and the pair of nuts 12. The bar 14 is mounted onto the pair of rods 10 via complementary screw threads, such that rotation of the rods 10, via the respective nuts 12, causes the bar 14 to move along the rods 10.

A mounting element 16 is attached (glued) to the nail of the thumb 2. The mounting element 16 comprises a clip into which the bar 14 engages (this can be seen in more detail in FIG. 1c).

Operation of the distraction splint 1 will now be described, with reference to FIGS. 1a, 1b, 1c and 1d.

To use the distraction splint 1 shown in FIGS. 1a, 1b, 1c and 1d, the mounting element 16 is attached (glued) to the nail of the thumb 2. The support 6 is placed over the thumb 2 to fit around the MCP joint and rest on the palm of the hand 4 at the base of the thumb 2.

The nuts 12 are adjusted (e.g. using the tool 18 shown in FIG. 1d) to move the bar 14 into position so that it engages with the clip of the mounting element 16. In this position, the nuts 12 can be further adjusted so to increase the distance between the mounting element 16 and the support 6, along the rods 10. This causes a biasing force to be applied between the mounting element 16 and the support 6, through the rods 10.

The biasing force is used to distract the carpometacarpal (CMC) joint at the base of the thumb 2. The nuts 12 can be adjusted to vary the magnitude of the biasing force, and thus the amount of distraction, that is applied.

The distraction of the CMC joint helps to relieve the symptoms of osteoarthritis and enable natural healing of the joint to occur, or may help to treat a fracture in the thumb 2. The pivotable mounting of the rods 10 in the support 6 allows the user to have some movement of their thumb 2 while wearing the splint 1. Furthermore, the bar 14 may be unclipped from the mounting element 16, by adjusting the nuts 12, should more dexterity be required temporarily. The splint 1 is then easily refitted in the manner described above.

FIGS. 2a and 2b show views of a distraction splint 101 according to another embodiment of the present invention.

The splint 101 comprises a support 106 that removably fits around the base of the thumb 102, around the metacarpophalangeal (MCP) joint. The support 106 also extends around the hand 104 of the person wearing the splint 101. The support 106 is made from an elastic material, containing multiple ventilation holes 108, that stretches to fit over and then conform to the shape of the hand 104.

A mounting element 116 is attached (glued) to the nail of the thumb 102. The mounting element 116 is attached to one part of a ratchet that engages with an adjustable member 110. The adjustable member 110 is mounted on the support 106 at the outside of the thumb 102.

The adjustable member 110 comprises an adjustable ratchet mechanism 114 that allows the distance between the support 106 and the mounting element 116 to be varied. The ratchet mechanism 114 retains the adjustable member 110 in its extended position to allow a biasing force to be applied between the support 106 and the mounting element 116.

Operation of the distraction splint 101 shown in FIGS. 2a and 2b is similar to the distraction splint 1 shown in FIGS. 1a, 1b, 1c and 1d.

First, the support 106 is placed over the thumb 102 to fit around the MCP joint and around the hand 104 at the base of the thumb 102 and palm. The mounting element 116 is attached (glued) to the nail of the thumb 102 and the two parts of the adjustment member 110 (including the ratchet mechanism 114) are assembled.

The two parts of the adjustment member 110 are moved away from each other, to increase the distance between the mounting element 116 and the support 106. The ratchet mechanism 114 retains the adjustment member 110 in this position and causes a biasing force to be applied between the mounting element 116 and the support 106, through the adjustment member 110.

The biasing force is used to distract the carpometacarpal (CMC) joint at the base of the thumb 102. The ratchet mechanism 114 can be adjusted to vary the magnitude of the biasing force, and thus the amount of distraction, that is applied.

FIGS. 3a and 3b show views of a distraction splint 201 according to another embodiment of the present invention.

The splint 201 comprises a support 206 that removably fits around the base of the thumb, around the metacarpophalangeal (MCP) joint. The support 206 is made plastic and contains multiple ventilation holes 208.

A mounting element 216 is attached (glued) to the nail of the thumb. The mounting element 216 is attached to one end of an adjustable member 210. The adjustable member 210 extends around the thumb, with its other end being mounted on the support 206.

The adjustable member 210 comprises an adjustable rotating ratchet mechanism 214 that allows the distance between the support 206 and the mounting element 216 to be varied. The ratchet mechanism 214 retains the adjustable member 210 in its extended position to allow a biasing force to be applied between the support 206 and the mounting element 216.

Operation of the distraction splint 201 shown in FIGS. 3a and 3b is similar to the distraction splint 1 shown in FIGS. 1a, 1b, 1c and 1d and the distraction splint 101 shown in FIGS. 2a and 2b.

First, the support 206 is placed over a thumb to fit around the MCP joint. The mounting element 216 is attached (glued) to the nail of the thumb.

The ratchet mechanism 214 of the adjustment member 210 is rotated to increase the distance between the mounting element 216 and the support 206. The ratchet mechanism 214 retains the adjustment member 210 in this position and causes a biasing force to be applied between the mounting element 216 and the support 206, through the adjustment member 210.

The biasing force is used to distract the carpometacarpal (CMC) joint at the base of the thumb. The ratchet mechanism 214 can be adjusted to vary the magnitude of the biasing force, and thus the amount of distraction, that is applied.

FIGS. 4a, 4b and 4c show views of a distraction splint 301 according to another embodiment of the present invention. The distraction splint 301 shown in FIGS. 4a, 4b and 4c shares a number of features with the distraction splint shown in FIGS. 1a, 1 b, 1c and 1d.

The distraction splint 301 comprises a support 306 that removably fits around the base of the thumb 302. The support 306 is moulded (e.g. to fit the hand 304 of the person) from plastic and is shaped to rest on the palm of the hand 304 at the base of the thumb 302. The support 306 is shaped such that it leaves the majority of the palm of the hand 304 free and such that it does not cover the metacarpophalangeal (MCP) joint, to avoid applying pressure to these parts of the hand 304.

The support 306 has multiple holes 308 formed through the thickness of the support 306. The holes 308 provide some ventilation to help prevent the support 306 causing the hand 304 to become sweaty. The support 306 is also shaped to allow space for blood vessels supply in the hand 304.

A pair of adjustable rods 310 is mounted on the support 306 either side of the thumb 302. The rods 310 each comprise a screw thread (in the form of an extended grub screw). The rods 310 each comprise a domed nut 318 that is fixedly attached to the lower end of the respective rod 310 (as can be seen in FIG. 4c in which the support 306 has been omitted to illustrate the domed nuts 318).

The domed nuts 318 are mounted pivotably to recesses in the support 306, with the support 306 comprising stoppers in the recesses to prevent the rods 310 from being moved substantially longitudinally (in a direction away from the base of the thumb 302). This helps to prevent the rods 310 from being extracted from the support 306. The depth of the recesses in the support 306 prevents the rods 310 from moving further into the support 306. The rods 310 are also mounted such that they are able to rotate freely (in the rotational direction the screw threads).

The other ends of the rods 310 (remote from the support 306) comprise a pair of hexagonal recesses that is formed in the pair of rods 310 respectively. The hexagonal recesses allow respective rods 310 to be rotated by means of an Allen key that fits into the hexagonal recess.

A bar 314 is mounted on and between the pair of rods 310, at the end of the pair of rods 310. The bar 314 is mounted onto the pair of rods 310 via complementary screw threads (e.g. in the form of respective nuts fixed (e.g. glued) into sockets in the sides of the bar 314), such that rotation of the rods 310, actuated by an Allen key, causes the bar 314 to move along the rods 310.

A mounting element 316, formed of a flexible material is attached (glued) to the nail of the thumb 302. The mounting element 316 comprises a slot into which a projection of the bar 314 engages (this can be seen in more detail in FIG. 4c).

Operation of the distraction splint 301 shown in FIGS. 4a, 4b and 4c is similar to the distraction splint 1 shown in FIGS. 1a, 1b, 1c and 1d.

The mounting element 316 is attached (glued) to the nail of the thumb 302. The support 306 is placed over the thumb 302 to fit around the MCP joint and rest on the palm of the hand 304 at the base of the thumb 302.

The rods 310 are adjusted (using an Allen key) to move the bar 314 into position so that its projection fits into the slot of the mounting element 316. In this position, the rods 310 can be further adjusted so to increase the distance between the mounting element 316 and the support 306, along the rods 310. This causes a biasing force to be applied between the mounting element 316 and the support 306, through the rods 310. The biasing force helps to retain the projection of the bar 314 in the slot of the mounting element 316.

The biasing force is used to distract the carpometacarpal (CMC) joint at the base of the thumb 302. The rods 310 can be adjusted to vary the magnitude of the biasing force, and thus the amount of distraction, that is applied.

FIGS. 5a-5e show views of a distraction splint 401 according to another embodiment of the present invention. The distraction splint 401 shown in FIGS. 5a, 5b, 5c, 5d and 5e shares a number of features with the distraction splint shown in FIGS. 1a, 1b, 1c and 1d. FIGS. 6a-6c, 7a-7c, 8a-8b and 9a-9b show more detailed views of the various components of the distraction splint 401 shown in FIGS. 5a-5e.

The distraction splint 401 comprises a support 406 made from a flexible material that removably fits around the base of the thumb 402. The support 406 is shaped such that it leaves the majority of the palm of the hand 404 free and such that it does not cover the metacarpophalangeal (MCP) joint, to avoid applying pressure to these parts of the hand 404.

The support 406 has multiple holes 408 formed through the thickness of the support 406. The holes 408 provide some ventilation to help prevent the support 406 causing the hand 404 to become sweaty. The support 406 is also shaped to allow space for blood vessels supply in the hand 404.

A pair of adjustable rods 410 is mounted on the support 406 either side of the thumb 402. The rods 410 each comprise a screw thread 411 and a (e.g. a domed, e.g. a ball) head 412 (e.g. the rods 410 are in the form of a screw 411 with a domed head 412) as can be seen in FIGS. 6c and 6d.

As shown in FIGS. 6a and 6b, the domed heads 411 are mounted pivotably to recesses in the support 406, with the support 406 comprising stoppers 415 in the recesses to prevent the rods 310 from being moved substantially longitudinally (in a direction away from the base of the thumb 402), e.g. the domed head 412 and the stoppers form a ball-and-socket joint. This helps to prevent the rods 410 from being extracted from the support 406. The depth of the recesses in the support 406 prevents the rods 410 from moving further into the support 406. The rods 410 are also mounted such that they are able to rotate freely (in the rotational direction the screw threads).

The other ends of the rods 410 (remote from the support 406) comprise a pair of hexagonal recesses 420 that is formed in the pair of rods 410 respectively. The hexagonal recesses allow respective rods 410 to be rotated by means of an Allen key that fits into the hexagonal recess 420.

Returning to FIG. 5a-5e in conjunction with FIGS. 7a-7c, a bar 414 is mounted on and between the pair of rods 410, at the end of the pair of rods 410. The bar 414 is mounted onto the pair of rods 410 via complementary screw threads 418. For example, the bar 414 comprises two apertures 417 positioned at the sides of the bar 414 where each aperture 417 comprises an integrally formed complementary screw thread 418 (e.g. complementary to the screw thread 411 of the rod 410). For example, the complementary thread 418 acts as the respective nut, integrally formed into aperture 417 in the sides of the bar 414, such that rotation of the rods 410, actuated by an Allen key, causes the bar 414 to move along the rods 410.

FIG. 7c shows a detailed view of the internal thread 418 of the bar 414. Both the rods 410 and bar 414 are made from a rigid material and may be 3D-printed.

FIGS. 8a and 8b show the mounting element 416, formed of a flexible material, which is attached (glued) to the nail of the thumb 402 (shown in FIGS. 5a-5c). The mounting element 416 comprises a slot 425 into which a projection of the bar 414 engages (this can be seen in more detail in FIG. 5d).

Operation of the distraction splint 401 shown in FIGS. 5a-5d is similar to the distraction splint 1 shown in FIGS. 1a, 1b, 1c and 1d.

The mounting element 416 is attached (glued) to the nail of the thumb 402. The support 406 is placed over the thumb 402 to fit around the MCP joint and rest on the palm of the hand 404 at the base of the thumb 402.

The rods 410 are adjusted (using an Allen key and hexagonal recess 420) to move the bar 414 into position so that its projection fits into the slot of the mounting element 416. In this position, the rods 410 can be further adjusted so to increase the distance between the mounting element 416 and the support 406, along the rods 410. This causes a biasing force to be applied between the mounting element 416 and the support 406, through the rods 410. The biasing force helps to retain the projection of the bar 414 in the slot of the mounting element 416.

The biasing force is used to distract the carpometacarpal (CMC) joint at the base of the thumb 402. The rods 410 can be adjusted to vary the magnitude of the biasing force, and thus the amount of distraction, that is applied.

It can be seen from the above that, in at least preferred embodiments, the invention provides a distraction splint for a human digit. The mounting element and the support are conveniently fitted to different parts of the human digit, allowing distraction to be performed. The mounting element and the adjustment member are physically separate components that releasably engage with each other, which helps to allow the splint to be partially disassembled. This provides the user with more comfort and allows them to partially remove the splint temporarily to gain greater dexterity.

Embodiments have been shown in the drawings with particular reference to the distraction of a thumb. However, those skilled in the art will appreciate that embodiments of the splint may be suitable for distracting a finger or toe.

1. An orthosis for distracting a human digit, the orthosis comprising:
a support for removably fitting to a hand or foot comprising the digit;
a mounting element for attaching to the distal end of the digit; and
an adjustable member extending between the support and the mounting element;
wherein the adjustable member and the mounting element are physically separate components;
wherein the adjustable member is arranged to releasably engage with the mounting element;
wherein the adjustable member is mounted to the support;
wherein the adjustable member is movable with respect to one or both of the support and the mounting element so to increase the distance between the support and the mounting element; and
wherein the orthosis is arranged such that the increase in distance between the support and the mounting element causes the adjustable member to exert a biasing force between the support and the mounting element so to distract the digit.

2. The orthosis as claimed in claim 1, wherein the orthosis is arranged to distract the first carpometacarpal joint.

3. The orthosis as claimed in claim 1, wherein the support is arranged for removably fitting proximal to a metacarpophalangeal joint; and
wherein the support comprises a sheath for removably fitting around the base of the digit.

4. (canceled)

5. The orthosis as claimed in claim 1, wherein the support is extended from the location at which the adjustable member is mounted on the support, in a direction away from the direction in which the adjustable member extends towards the mounting element.

6. The orthosis as claimed in claim 1, wherein the support comprises a plurality of holes across the surface of the support.

7. The orthosis as claimed in claim 1, wherein the mounting element is arranged to be attached non-surgically to the distal end of the digit; and
wherein the mounting element comprises a place for attaching to the nail of the digit.

8. (canceled)

9. The orthosis as claimed in claim 1, wherein the adjustable member comprises a screw thread.

10. The orthosis as claimed in claim 1, wherein the adjustable member comprises two rods that extend between the support and the mounting element.

11. The orthosis as claimed in claim 10, the adjustable member comprises two knobs or sockets for actuating the two rods respectively of the adjustable member to increase the distance between the support and the mounting element.

12. The orthosis as claimed in claim 10, wherein the adjustable member comprises a bar that extends between the two rods, wherein the bar is arranged to releasably engage with the mounting element;
wherein the position of the bar is adjustable relative to the rods; and
wherein the two rods comprise screw threads and the bar comprises complementary screw threads for adjusting the position of the bar relative to the rods.

13-14. (canceled)

15. The orthosis as claimed in claim 1, wherein the adjustable member and the mounting element comprise respective complementary engaging surfaces, wherein the adjustable member and the mounting element are arranged to releasably engage with each other at the complementary engaging surfaces.

16. The orthosis as claimed in claim 1, wherein the adjustable member and the mounting element comprise complementary releasable fasteners.

17. The orthosis as claimed in claim 1, wherein the adjustable member and the mounting element are arranged such that the biasing force acts in an opposite direction to the direction in which the adjustable member and the mounting element release from each other.

18. The orthosis as claimed in claim 1, wherein the adjustable member and the mounting element are arranged such that the support is able to be removed from the digit when the adjustable member and the mounting element are released from each other.

19. The orthosis as claimed in claim 1, wherein the adjustable member is mounted to the support such that longitudinal movement of the adjustable member is substantially prevented.

20. The orthosis as claimed in claim 1, wherein the adjustable member is pivotably mounted to the support.

21. The orthosis as claimed in claim 1, wherein at least a part of the adjustable member is rotatable with respect to one or both of the support and the mounting element so to increase the distance between the support and the mounting element.

22. The orthosis as claimed in claim 1, wherein the adjustable member comprises two parts having complementary screw threads, wherein the two parts are rotatable relative to each other to increase the distance between the support and the mounting element.

23. The orthosis as claimed in claim 1, wherein the orthosis comprises an actuator that is fixedly attached to the adjustable member for actuating the adjustable member.

24. The orthosis as claimed in claim 1, wherein the orthosis is arranged such that the biasing force is applied in the direction between the support and the mounting element.
